(19) United States
(12) Patent Application Publication
(10) Pub. No.: US 2023/0008536 A1
(43) Pub. Date: Jan. 12, 2023

(54) X-RAY DETERMINATION OF AN OBJECT’S LOCATION WITHIN A BODY

(71) Applicant: ADAPTIX LTD, Begbroke (GB)

(72) Inventors: Gil Travish, Begbroke (GB); Zena Patel, Begbroke (GB)

(21) Appl. No.: 17/946,362

(22) Filed: Sep. 16, 2022

Related U.S. Application Data
(63) Continuation of application No. PCT/IB2021/051995, filed on Mar. 10, 2021.

(30) Foreign Application Priority Data
Mar. 16, 2020 (GB) 2003769

(51) Int. Cl. A61B 6/02; A61B 6/00

(52) U.S. Cl. CPC A61B 6/025; A61B 6/4007; A61B 6/4085

(57) ABSTRACT

Digital tomosynthesis (DT) gives better diagnostic information than 2D X-ray, rivalling CT. However, tomosynthesis reconstruction requires sophisticated algorithms and a powerful computer, and can take several minutes to complete. The present invention takes a single x-ray image of a body 50 using multiple sources. In normal tomography and tomosynthesis imaging, such overlapping cones would lead to un-reconstructable data as significant overlap, in general, can’t be deconvolved and is not soluble. However, here, for the detection and localization of dense, compact objects 40, a location of an object 40 may be determined in three spatial dimensions from a single two-dimensional image. That is, processor-intensive reconstruction of a three-dimensional volume may be avoided.

### RELATED APPLICATIONS

This application claims the benefit under 35 U.S.C. § 120, and is a continuation, of co-pending International Application PCT/IB2021/051995, filed Mar. 10, 2021 and designating the US, which claims priority to GB Application 2003769.3, filed Mar. 16, 2020, such GB Application also being claimed priority to under 35 U.S.C. § 119. These GB and International applications are incorporated by reference herein in their entireties.

### Field

The present invention relates generally to a system and a method of determining a location of an object within a body and finds particular, although not exclusive, utility in identifying the location of shrapnel in trauma patients.

### BACKGROUND

Foreign objects, usually small and dense, can be easy to see on an x-ray but difficult to precisely locate on 2D radiographs. In healthcare, where the objects might be embedded in a body, radiologists often rely on “landmarks” (adjacent anatomy) to locate the objects. However, for surgical planning or trauma assessment this sort of localization can be slow and imprecise. Such foreign objects can also be hidden behind bone or implants, and hence not easily detected on an x-ray. In nonhealthcare settings, similar considerations apply to foreign objects or contaminants in food, manufactured goods, etc. Such objects can become embedded as a result of industrial production (e.g. a metal fragment in a food processing plan) or intentionally hidden (e.g. sabotage of a machine or malicious modification of electronics).

### SUMMARY

For explanatory purposes, the following disclosure focuses on healthcare and the case of shrapnel. However, similar explanations and considerations apply to any small, dense matter imbedded in another (less x-ray opaque) object.

Shrapnel injuries from bomb blasts are increasing in number and are a major cause of severe trauma and death in military personnel and civilians in conflict zones. The superficial appearance of shrapnel entry wounds can be deceptive; therefore, the casualty may suffer further injury if the shrapnel is not detected quickly. The ability to detect shrapnel in-the-field would be useful for the first response medical personnel to diagnose the patient. Similarly, in civilian settings, trauma from penetration wounds and projectiles from domestic and industrial explosions can cause occult injuries with foreign objects imbedding into a patient.

Shrapnel can and does migrate inside the body. The ability to detect shrapnel before moving the patient (i.e. in-the-field) will reveal if the patient’s injuries make them suitable or unsuitable to be moved. If the shrapnel is too close to a major organ, then the movement of the patient should be avoided if possible. The ability to detect shrapnel in the field would prevent further injury/death.

Proper in-the-field triage also leads to better utilization of limited medical resources. One problem for medical staff at a civilian hospital following detonation of an explosive device is the sudden creation of a large number of patients. An in-the-field detection device can decrease the number of casualties sent to a hospital. It can also aid military personnel in the decision to remove someone from combat. The importance of accurate triage has been well-documented as a survival determinant for critically injured casualties.

There are several diagnostic tools available for shrapnel detection, including conventional 2D X-ray imaging and computed tomography (CT) imaging. These tools are used in a hospital setting before (or during) surgery for shrapnel removal, or directly following the event in which the shrapnel injury occurred.

2D X-rays only give a shadowgram and hence all depth information is lost.

A CT scan is commonly performed before surgery to locate the shrapnel for removal as this type of scan provides a precise location. This can be very useful in a hospital setting, but (due to the bulky size of the CT equipment and the complex use and readout) is not practical for in-the-field use.

Digital tomosynthesis (DT), a low-dose and inexpensive imaging technique where X-rays are emitted from a limited range of angles to derive 3D data, has been shown to give better diagnostic information than 2D X-ray, rivalling CT. In digital tomosynthesis images, information is reconstructed into a set of “slices” that preserve depth locations. However, tomosynthesis reconstruction requires sophisticated algorithms and a powerful computer. Reconstructions can take several minutes. In addition, metal artefacts, while normally well tolerated in digital tomosynthesis compared to computed tomography, can still cause image formation problems. Significant metal can lead to “photon starvation” and cause reconstruction problems that complicate reading of the radiographs.

To date, the pre-hospital diagnosis and care for casualties with bomb blast injuries does not involve imaging the casualty for embedded shrapnel.

According to a first aspect of the present invention, there is provided a method of determining a location of an object within a body, the method comprising the steps of: providing an x-ray detector panel; providing a first x-ray emitter at a first location relative to the x-ray detector panel, the first x-ray emitter configured to emit a first cone of x-rays therefrom such that the first cone of x-rays impinges the x-ray detector panel; providing a second x-ray emitter at a second location relative to the x-ray detector panel, the second location spaced from the first location, the second x-ray emitter configured to emit a second cone of x-rays therefrom such that the second cone of x-rays impinges the x-ray detector panel; activating the first x-ray emitter and the second x-ray emitter concurrently to produce a single two-dimensional image at the detector panel indicative of absorption of the first and second cones of x-rays by a body placed therebetween; determining in the single two-dimensional image respective first and second positions of an object projected by the first and second cones of x-rays respectively; and establishing a site of the object relative to the x-ray detector panel from the first location, second location, first position and second position.

In this way, a location of an object may be determined in three spatial dimensions from a single two-dimensional image. That is, processor-intensive reconstruction of a three-dimensional volume may be avoided.

In normal tomography and tomosynthesis imaging, such overlapping cones would lead to un-reconstructable data as significant overlap, in general, can’t be deconvolved and is not soluble. However, here, for the detection and localization of dense, compact objects such overlap can be resolved.

The location may be an approximate location, for example to within an accuracy and/or a precision of one, two or three significant figures.

The body may comprise a human and/or animal body. Alternatively or additionally, the body may comprise any other form of mass (comprising solid and/or liquid), such as manufactured components, biological samples, etc.

The location being within a body may comprise the location being at or below a surface of the body.

The x-ray detector panel may comprise a conventional digital or analogue x-ray detector panel.

The x-ray emitter(s) may comprise any conventional x-ray emitter; that is, capable of emitting x-rays. The cone(s) of x-rays may be defined by the path of x-rays from the emitter(s), such that emission of x-rays may be substantially rotationally symmetric about a respective emission axis, and such that emission of x-rays may fall off rapidly beyond some threshold emission angle from the emission axis. That is, x-rays may be emitted within an emission angle from the emission axis, and x-ray emission may be prohibited beyond the emission angel from the emission axis. The emission angle may be between 5 degrees and 80 degrees, in particular between 10 degrees and 40 degrees, for example 20 degrees.

The x-ray emitter(s) may be located such that at least some of the x-rays emitted in the respective cone(s) fall on the x-ray detector panel. That is, the cone may entirely fall on the detector panel, or may only partially fall on the detector panel.

The second location may be spaced from the first location by a predefined spacing, and/or the spacing may be dynamically determined.

The predefined spacing may comprise a spacing integral to the emitters; that is, each emitter may be part of a single emitter panel, the emitter panel comprising an array of such emitters permanently fixed with respect to each other. Two of these emitters may be selected at any given time to be the first and second emitters, and such selection defines the spacing between them (based on manufacturing specifications, etc.). Alternatively or additionally, the predefined spacing may comprise a user-set spacing, chosen in view of the geometry of the body being examined.

Dynamically determining the spacing may comprise locating each of the first and second emitters in a desired location and then measuring (either automatically or manually) the spacing between them.

Similarly, a first location relative to the x-ray detector panel may be a predefined location, and/or the location relative to the x-ray detector panel may be dynamically determined in a similar way to that discussed above with respect to the spacing. For example, the first emitter may be at a fixed distance from the detector panel, or may be at a variable distance. Alternatively or additionally, the first emitter may be translatable (for example in one or two dimensions) in a plane parallel to the detector panel, or may be fixed within such a plane. Similarly, the second emitter may be fixed or translatable.

Activating the x-ray emitter(s) may comprise emitting x-rays therefrom.

Concurrently may mean substantially at the same time, for example a first x-ray from the second emitter may impinge the detector before a final x-ray of the first emitter (or vice versa). However, in preferred embodiments it is not necessary for the first x-ray from each emitter to be emitted at the same time, although this is contemplated. Alternatively or additionally, concurrently may mean that the x-rays from the first emitter and x-rays from the second emitter are emitted within an exposure period over which the x-ray detector panel integrates the single two-dimensional image (i.e. an integration time). The integration time may be between 0.5 seconds and 15 seconds, in particular between 1 second and 10 seconds, more particularly between 2 seconds and 8 seconds. In this way, a first x-ray from the second emitter may impinge the detector after a final x-ray of the first emitter (or vice versa), but a single two-dimensional image is still produced.

A single two-dimensional image may comprise a photographic-like image, or more preferably a digital image file. X-rays arriving at the detector panel may have been attenuated by the body placed therebetween, thus the intensity profile in the image is indicative of absorption qualities of the material therein.

Determining respective positions of an object may comprise identifying a one- or two-dimensional co-ordinate of an object, for example in a cartesian and/or polar geometry.

Establishing a site of the object relative to the x-ray detector panel may comprise calculating a distance of the object from the detector panel. In particular, trigonometric principles may be used to determine the distance.

Determining a location of an object may comprise determining a one-, two- or three-dimensional co-ordinate of an object, for example in a cartesian, cylindrical and/or spherical geometry.

The x-ray emitters may form part of one or more distributed x-ray sources and/or x-ray emitter panels (e.g. array(s) of x-ray emitters). The or each emitter panel may be flat, curved, etc.

The method may further comprise providing output to an operator.

The output may comprise a 3D model showing locations of objects embedded therein. This is clear to understand and interpret by medical personnel without the need for a specially trained radiographer. Alternatively or additionally, the output may be a list of object locations (e.g. in some co-ordinate system).

The object may be a unitary item; that is, the object may be a single distinct element having a length, breadth, depth and/or shape.

The object may be part of a composite component. That is, the object may be a part of a larger object. The object may be, for example, a corner of a solid piece of foreign item. In this way, the above method may be performed on four corners of a composite component having a quadrilateral profile, thereby allowing an operator to determine the extent and depth of the composite component.

In practice, the term object may be used to refer to a point (i.e. having no length, breadth, depth and/or shape), and the location of a composite component may be identified by determining the locations of a plurality of point objects around the composite component’s periphery.

The method may further comprise: providing a third x-ray emitter at a third location relative to the x-ray detector panel, the third location spaced from the first and second locations and non-co-linear with the first and second locations, the third x-ray emitter configured to emit a third cone of x-rays therefrom such that the third cone of x-rays impinges the x-ray detector panel; activating the third x-ray emitter concurrently with the first x-ray emitter and the second x-ray emitter to produce the single two-dimensional image at the detector panel indicative of absorption of the first, second and third cones of x-rays by a body placed therebetween; determining in the single two-dimensional image a third position of the object projected by the third cone of x-rays; and establishing a site of the object relative to the x-ray detector panel from the first location, second location, third location, first position, second position and third position.

In this way, localization of the object can be improved. Additional x-ray emitters may be employed in a similar manner to the third emitter, such that a single two-dimensional image is produced. There may be four, five, six, seven, eight, nine or more such emitters.

Each emitted cone should be arranged to illuminate each voxel (volume element) under consideration. Additional emitters may be used to provide additional accuracy or overcome imaging complications from bones or overlapping shrapnel pieces.

Performing the illumination by each x-ray emitter concurrently reduces image acquisition time, and artefacts introduced due to body (e.g. patient) movement between exposures. However, in some circumstances some additional emitters (over and above the first and second emitters) may be activated non concurrently with the fist and second emitters in order to provide a clearer data set, for example for subsequent tomosynthesis reconstruction.

Shrapnel or other substantially x-ray opaque objects appear in the single two-dimensional image as bright spots, regions or rings (depending on the distance to the detector and size of the object), as the X-rays are absorbed by it whereas the X-rays will pass through surrounding tissue (such as soft biological tissue).

Optionally, using an x-ray detector positioned on an opposite side of the subject (e.g. patient or other body) to an x-ray source, an overall image of a subject may first be taken to detect any candidate objects. Shrapnel or other substantially x-ray opaque objects appear in the image as bright regions, as x-rays are absorbed by them whereas x-rays will pass through natural tissue. This will provide an approximate position of the candidate objects in the x-y plane (i.e. parallel to the detector panel) displayed on an image of the patient’s body in a similar manner to planar X-ray.

For example, using a flat panel array, a subset of emitters distributed equally throughout the region of interest is switched on to get a single exposure of the body, and the X-Y location of any objects. If this image shows discrete objects, then a cluster of emitters (e.g. 2, 3, 4 or more) above each piece of shrapnel may be switched on, and the Z location may be determined by trigonometry. A 3D image of the body may then be made available to operator, performing the imaging. This may show the location in three dimensions of any objects in relation to structures in the body. In a medical context, this will enable a medic to make a go/no-go decision in removing the patient from the field.

According to a second aspect of the present invention, there is provided a system for determining a location of an object within a body, the system comprising: an x-ray detector panel; a first x-ray emitter at a first location relative to the x-ray detector panel, the first x-ray emitter configured to emit a first cone of x-rays therefrom such that the first cone of x-rays impinges the x-ray detector panel; a second x-ray emitter at a second location relative to the x-ray detector panel, the second location spaced from the first location, the second x-ray emitter configured to emit a second cone of x-rays therefrom such that the second cone of x-rays impinges the x-ray detector panel; a control device configured to activate the first x-ray emitter and the second x-ray emitter concurrently to produce a single two-dimensional image at the detector panel indicative of absorption of the first and second cones of x-rays by a body placed therebetween; an image analyzer configured to determine in the single two-dimensional image respective first and second positions of an object projected by the first and second cones of x-rays respectively; and a processor configured to establish a site of the object relative to the x-ray detector panel from the first location, second location, first position and second position.

The control device, image analyzer and processor may be realized in the form of software and/or a computer system.

The image analyzer may be configured to perform image recognition on the single two-dimensional image to automatically identify regions indicative of high contrast and/or high attenuation. ‘High’ may mean near total absorption of x-rays, or at least an order of magnitude (e.g. 2, 5, 10, 20 or 50 times) higher than absorption by surrounding tissue in the body.

The image analyzer may be configured to perform image recognition on regions of the single two-dimensional image in which high contrast and/or high attenuation have been identified, in order to assign features in the region as due to emission from the first and/or second emitters.

Such recognition may be performed automatically, for instance using a pre-defined threshold, and/or by using machine learning techniques. Alternatively or additionally, such recognition may be performed manually.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 shows a simplified view of a physical arrangement of components and a resultant image due to three emitters.

FIG. 2 shows a resultant image due to nine emitters.

FIG. 3 shows a simplified representation of the location of shrapnel in a patient.

FIG. 4 shows one possible geometry.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other. For instance, wireless connectivity is contemplated.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment,figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

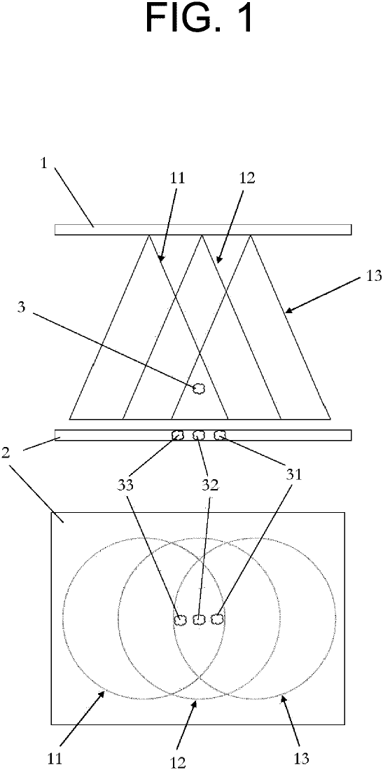

FIG. 1 shows in the upper portion an emitter array 1, a detector panel 2, and an object 3 disposed therebetween. Three respective cones 11, 12, 13 of x-ray radiation are shown emanating from three respective x-ray emitters (not shown) in the emitter array 1. Three respective images 31, 32, 33 are shown on the detector panel 2, due to the three respective cones 11, 12, 13.

In the lower portion of FIG. 1, the three respective images 31, 32, 33 are again shown as they would appear on the detector panel 2. The projections of the cones 11, 12 13 onto the detector panel 2 are also indicated.

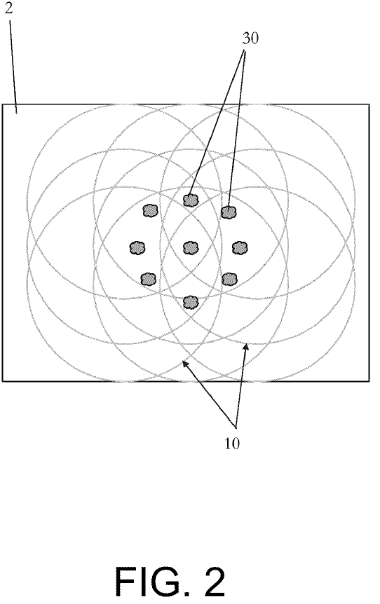

FIG. 2 shows a resultant image, similar to that shown in the lower portion of FIG. 1, due to nine emitters of the emitter array 1 being activated to produce nine images of the object 3 on the detector panel 2, with the corresponding cone projections 10. It should be noted that the geometry of FIG. 2 is different to that of FIG. 1, resulting in a different spacing of the images 30 on the detector panel 2 with respect to the cone projections 10.

It should also be noted that FIGS. 1 and 2 show the resultant image due to a well-defined, spatially small object, compared to the cone projection and/or overlap. Such resultant images show multiple discrete instances of the single object’s projection. However, where the object to be imaged is large compared to the cone projection and/or overlap, the resultant image would not show multiple discrete instances of the object’s projection, but instead the single object’s projections may overlap one another. In circumstances where a relatively large number of emitters are used (e.g. four, five, six or more), in particular, the resultant image of the object may appear indistinct and/or blurred, particularly at the edges. In such cases, the site of the object may still be established by similar trigonometric procedures, for example taking account of an amount of blurring at a periphery of the object’s projection.

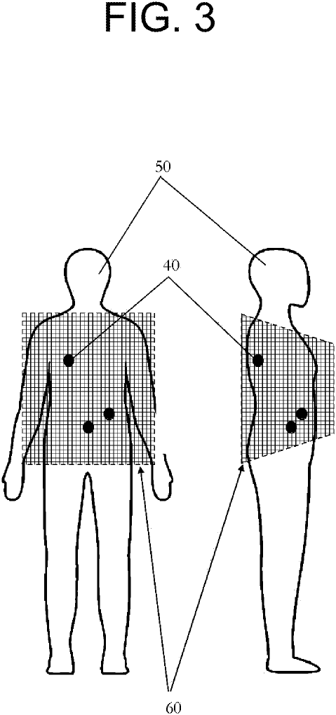

FIG. 3 shows a simplified representation of the location of shrapnel 40 in a patient 50, shown in the coronal plane to the left and in the sagittal plane to the right. A grid 60 is overlaid in each view to show the regions covered by the x-ray cones (not shown), and to help identify accurate locations.

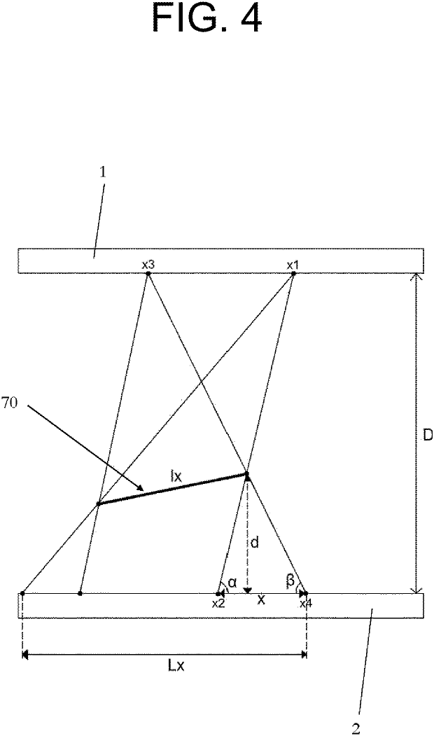

FIG. 4 shows one possible geometry used between the emitter array 1 and the detector panel 2, shown parallel and spaced apart a distance D. An elongate object 70 is shown therebetween, illuminated by two respective emitters shown at x1 and x3. An image of the elongate object 70 is indicated on the detector panel 2 by Lx.

To calculate the depth of the elongate object 70 where only two emitters are used, first the angles α and β in FIG. 4 may be calculated:

$$α = a r c s i n \left[\frac{D}{{\left({x}_{1} - {x}_{2}\right)}^{2} + {D}^{2}}\right]$$

$$β = a r c s i n \left[\frac{D}{{\left({x}_{3} - {x}_{4}\right)}^{2} + {D}^{2}}\right]$$

This may be repeated for any edge points of the elongate object 70. From these angles the depth, d, of the elongate object 70 at the edge point can be calculated using:

$$d = \frac{x}{\frac{1}{t a n \left(α\right)} + \frac{1}{t a n \left(β\right)}}$$

The depth may also be calculated for each edge point on the elongate object 70. The length of the elongate object can be calculated using the depth and the total length of the dark spot on the detector:

$${l}_{x} = {L}_{x} \frac{D - d}{d}$$

Other triangulation methods are contemplated. For instance, shift and add methods are well known from tomosynthesis.

The X-ray panel source could also be multi-paneled or curved such that it could be on wheels and rolled over the patient. The geometry would be modified from the original method in this instance and a modified method of triangulation used, as would be well understood by the skilled person.

1. A method of determining a location of an object within a body, the method comprising the steps of:
providing an x-ray detector panel;
providing a first x-ray emitter at a first location relative to the x-ray detector panel, the first x-ray emitter configured to emit a first cone of x-rays therefrom such that the first cone of x-rays impinges the x-ray detector panel;
providing a second x-ray emitter at a second location relative to the x-ray detector panel, the second location spaced from the first location, the second x-ray emitter configured to emit a second cone of x-rays therefrom such that the second cone of x-rays impinges the x-ray detector panel;
activating the first x-ray emitter and the second x-ray emitter concurrently to produce a single two-dimensional image at the detector panel indicative of absorption of the first and second cones of x-rays by a body placed therebetween;
determining in the single two-dimensional image respective first and second positions of an object projected by the first and second cones of x-rays respectively; and
establishing a site of the object relative to the x-ray detector panel from the first location, second location, first position and second position, wherein the site of the object is determined in three spatial dimensions from the single two-dimensional image.

2. The method of determining a location of an object within a body of claim 1, the method further comprising the steps of:
providing a third x-ray emitter at a third location relative to the x-ray detector panel, the third location spaced from the first and second locations and non-co-linear with the first and second locations, the third x-ray emitter configured to emit a third cone of x-rays therefrom such that the third cone of x-rays impinges the x-ray detector panel;
activating the third x-ray emitter concurrently with the first x-ray emitter and the second x-ray emitter to produce the single two-dimensional image at the detector panel indicative of absorption of the first, second and third cones of x-rays by a body placed therebetween;
determining in the single two-dimensional image a third position of the object projected by the third cone of x-rays; and
establishing a site of the object relative to the x-ray detector panel from the first location, second location, third location, first position, second position and third position.

3. A system for determining a location of an object within a body, the system comprising:
an x-ray detector panel;
a first x-ray emitter at a first location relative to the x-ray detector panel, the first x-ray emitter configured to emit a first cone of x-rays therefrom such that the first cone of x-rays impinges the x-ray detector panel;
a second x-ray emitter at a second location relative to the x-ray detector panel, the second location spaced from the first location, the second x-ray emitter configured to emit a second cone of x-rays therefrom such that the second cone of x-rays impinges the x-ray detector panel;
a control device configured to activate the first x-ray emitter and the second x-ray emitter concurrently to produce a single two-dimensional image at the detector panel indicative of absorption of the first and second cones of x-rays by a body placed therebetween;
an image analyzer configured to determine in the single two-dimensional image respective first and second positions of an object projected by the first and second cones of x-rays respectively; and
a processor configured to establish a site of the object relative to the x-ray detector panel from the first location, second location, first position and second position.
